(19) United States
(12) Patent Application Publication
(10) Pub. No.: US 2006/0058799 A1
(43) Pub. Date: Mar. 16, 2006

(54) Ligament repair apparatus and method

(76) Inventors: Robert Elson, Los Altos Hills, CA (US); Daniel Irwin Jacobs, Palo Alto, CA (US); Kyle Naydo, Mountain View, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/056,817

(22) Filed: Feb. 10, 2005

Related U.S. Application Data
(60) Provisional application No. 60/543,633, filed on Feb. 10, 2004.

(51) Int. Cl. A61B 17/58

(52) U.S. Cl. 606/72

(57) ABSTRACT

Method and apparatus for securing first and second bones together by forming a cavity in the first and second bones, with the cavity including a shaped first anchor portion in the first bone and a shaped second anchor portion in the second bone, with a channel extending therebetween, fixating a first tissue anchor onto a first portion of tissue, fixating a second tissue anchor onto a second portion of the tissue, and inserting the first tissue anchor into the cavity first anchor portion and the second tissue anchor into the cavity second tissue anchor, wherein the tissue first and second portions are secured to the first and second bone, and the tissue extends along the channel. The tissue will form a strong biological construct with the sidewalls of the channel, whereby the tissue anchors may optionally dissolve away.

This application claims the benefit of U.S. Provisional Application No. 60/543,633, filed Feb. 10, 2004.

### FIELD OF THE INVENTION

The present invention relates to ligament repair, and more particularly to a method and apparatus that provides standardized attachment points for attaching ligaments to bone that enhances bone association and ligament repair.

### BACKGROUND OF THE INVENTION

Presently it can be difficult to repair injured ligaments that connect between separate bones. For example, when the ligament connecting the scaphoid and lunate bones in the wrist is injured, where the ligament is torn or ruptured from the bone causing the bone positions to disassociate, the injury is often repaired by simply fusing these bones together, where mobility between these bones is permanently lost. Instead of bone fusion, these bones can be reconnected together in a mobile fashion by grafting soft tissue on the surfaces of the bone. However, the strength of the soft tissue bond can be weak, and the ideal separation between these bones can be hard to achieve. Tissue grafts can have high failure or re-injury rates, even if the bones are pinned together during the initial healing process.

There is a need for a ligament repair apparatus and method that reliably connects bones together, that promotes bone to connective tissue healing to create a permanent reliable bone/tissue/bone fixation, that simplifies the actual operative techniques to implement, and that maintains bone mobility even during the healing process.

### SUMMARY OF THE INVENTION

The present invention solves the aforementioned problems by providing tissue anchors that crimp or swag onto the ends of connective tissue, a compression assembly for ensuring the tissue anchors are separated along the tissue by the desired distance, and a drilling template to ensure the bone cavity is formed with the correct dimensions. The present invention essentially transforms non-standard sized tissue and standardizes its dimensions for insertion into bone cavities of predetermined dimensions.

The anchor system of the present invention, for fixating portions of tissue in a bone cavity, includes a pair of tissue anchors each having an aperture, wherein the aperture includes side walls for receiving and exerting a fixating force onto a portion of the tissue.

In another aspect of the present invention, an anchor system for securing first and second bones together utilizing tissue having first and second portions and utilizing a bone cavity having a first anchor portion formed in the first bone and a second anchor portion formed in the second bone and a channel extending between the first and second anchor portions, includes a first tissue anchor dimensioned for insertion into the cavity first anchor portion, the first tissue anchor including a first aperture in which the first portion of the tissue is fixated, and a second tissue anchor dimensioned for insertion into the cavity second anchor portion, the second tissue anchor including a second aperture in which the second portion of the tissue is fixated, wherein the first and second bones are secured together by the first and second tissue anchors inserted in the cavity first and second anchor portions, and by the tissue extending between the first and second tissue anchors and through the channel.

In yet one more aspect of the present invention, a method of securing first and second bones together with tissue includes forming a cavity in the first and second bones, the cavity including a first anchor portion formed in the first bone, a second anchor portion formed in the second bone, and a channel extending between the first and second anchor portions, fixating a first tissue anchor onto a first portion of the tissue, fixating a second tissue anchor onto a second portion of the tissue, and inserting the first tissue anchor into the cavity first anchor portion and the second tissue anchor into the cavity second anchor portion such that the tissue extends along the channel.

Other objects and features of the present invention will become apparent by a review of the specification, claims and appended figures.

### BRIEF DESCRIPTION OF THE DRAWINGS

FIG. 1 is a perspective view of the tissue anchor assembly of the present invention.

FIG. 2 is a perspective view of the tissue anchor of the present invention.

FIG. 3 is a top view of the bone cavity formed to receive the tissue anchor assembly of the present invention.

FIG. 4 is a perspective view showing the formation of the tissue anchor using a mold insert.

FIGS. 5A and 5B are perspective views of the compression plates of the present invention. FIGS. 6A to 6D are perspective views illustrating the positioning plates of the present invention.

FIG. 7A and 7B are perspective views of the compression assembly of the present invention.

FIGS. 8A and 8B are perspective views of the drilling template of the present invention.

FIG. 9 is a top view illustrating the tissue anchor assembly implemented in the bone cavity.

FIG. 10 is a perspective view of the tissue carrier of the present invention.

FIG. 11 is a top view illustrating a scalloped surface of the tissue anchor assembly implemented in the bone cavity for use with an interference screw.

### DETAILED DESCRIPTION OF THE PREFERRED EMBODIMENTS

The present invention is a tissue anchor assembly and method of implementation of the same that securely connects two bones together with tissue while preserving mobility and promoting healing. The present invention is described in the context of reconstructing the ligament complex between the scaphoid and lunate carpal bones using harvested donor ligament tissue, but any bones can be connected together using the present invention, with any appropriate organic or inorganic connective tissue.

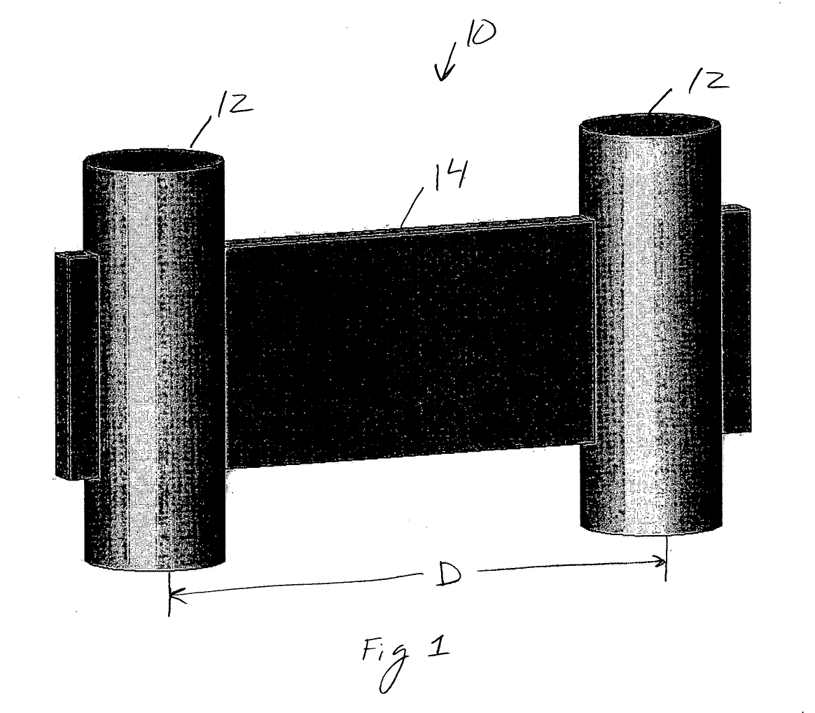

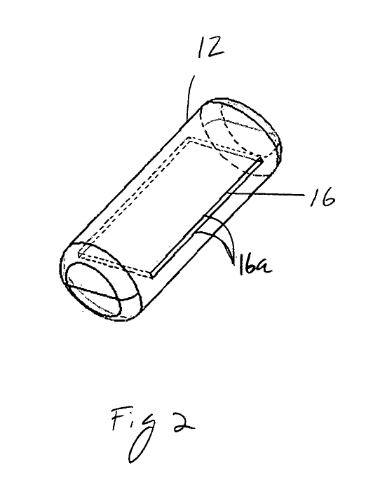

FIG. 1 illustrates the tissue anchor assembly 10 of the present invention, which includes a pair of tissue anchors 12 fixated to the ends of tissue 14. Tissue 14 can be any appropriate organic or inorganic, synthetic or natural, connective or muscular tissue, and/or any combinations thereof. Each of the tissue anchors 12 is a generally cylindrically shaped rigid member with an aperture 16 (e.g. a thin slot) formed therethrough, as shown in FIG. 2. Tissue anchors 12 may be made of any appropriate biocompatible material (preferably a polymer or copolymer), which also may be bio-absorbable, osteoconductive, and/or osteoinductive.

Each end of the tissue 14 is inserted into the slot (aperture) 16 of one of the tissue anchors 12, and then the tissue anchors 12 are crimped down (swaged) onto the tissue 14 so that sidewalls 16a of slot 16 exert a tissue fixating force onto the tissue to create a compressive lock that secures the tissue 14 in slots 16. The tissue anchors 12 are separated by a predetermined distance D, and provide a standardized shape for connecting the ends of tissue 14 to bone.

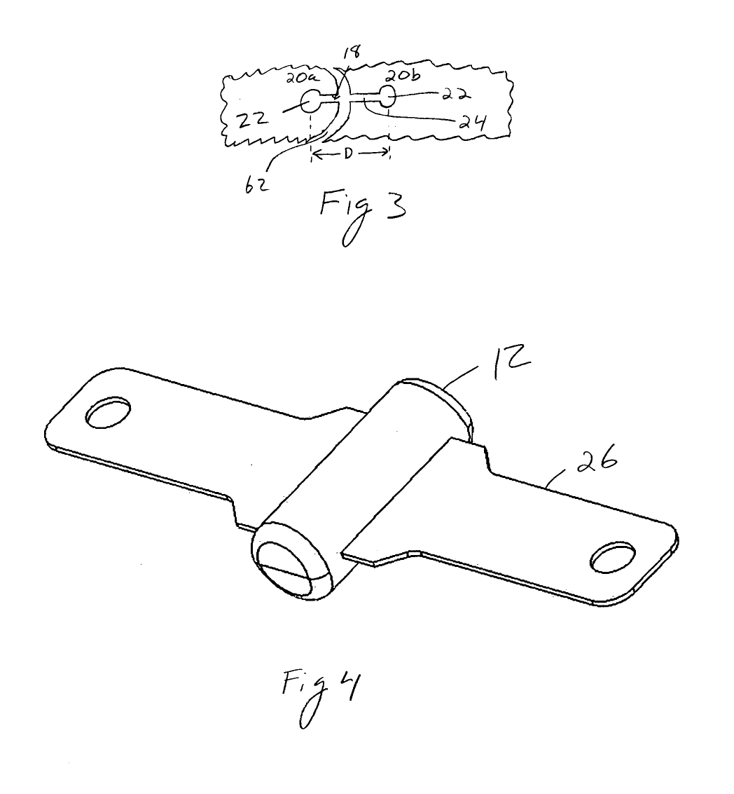

FIG. 3 illustrates the bone cavity 18 in which the tissue anchor assembly 10 is inserted. The bone cavity 18 is formed in the bones 20a and 20b which are to be connected together (separated by cartilage 62) using conventional bone drilling and channeling techniques. Bone cavity 18 includes a pair of cylindrical holes 22 (one in each of the bones 20a/b) which are preferably sized to match the size the tissue anchors 12. A channel 24 extends between the cylindrical holes 22 and is sized to receive the tissue 14. With the bones 20a/b ideally positioned with the desired separation (if any), holes 22 are separated by the distance D.

To anchor the bones 20a/b together, the tissue anchors 12 are inserted into the holes 22, such that the tissue 14 is inserted into the channel 24. Ideally, the tissue 14 and channel 24 are dimensioned to gently press the tissue 14 against the bone sidewalls of channel 24 to promote the healing of the tissue 14 to the bones 20a/b, and to ultimately result in a strong biological construct therebetween (without any excessive forces sufficient to cause bone erosion, tissue necrosis, etc.). Once the biological construct is formed between tissue 14 and bones 20a/b, tissue anchors 12 are no longer necessary. Thus, tissue anchors 12 can be made of a bio-absorbable material that dissolves after the healing period has ended and bone regeneration fills the void left from implanting the anchors.

Tissue Anchor Swaging

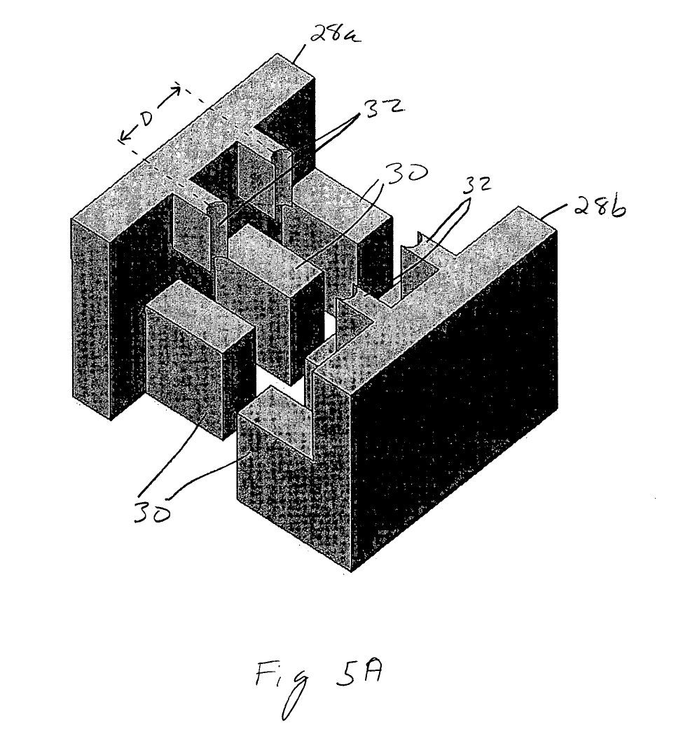

The tissue anchors 12 are preferably swaged onto the tissue ex-vivo in the following manner. First, the tissue anchors 12 are formed via molding with the slots 16 preferably having the smallest possible width. FIG. 4 illustrates one of the tissue anchors 12 molded around a very thin mold insert 26, which leaves slot 16 in tissue anchor 12 after being removed therefrom. The slots 16 are then manually expanded in width, for example, by driving a mandrel through each slot 16 to expand it. Depending upon the material used to form tissue anchors 12, the slots therein can be expanded while the tissue anchor material is at room temperature, or can be expanded while in an excited state (“excite expanded”, where the molecules of the tissue anchor material have been sped up). The excited state can be achieved by, for example, subjecting the tissue anchor material to heat (e.g. via conduction), ultrasonic waves, radiation (e.g. visible, ultraviolet, and/or infrared light from a laser, RF, etc.) and so on.

After the tissue 14 is inserted into slots 16, the tissue anchors 12 are mechanically compressed by a swaging or crimping action so that each tissue anchor 12 (via i.e. aperture sidewalls 16a) exerts a tissue fixation force that prevents the tissue 14 from sliding out of slot 16. Depending upon the material used to form tissue anchors 12, they can be compressed at room temperature, or can be compressed while in an excited state (“excite compressed”, where the molecules of the tissue anchor material have been sped up during compression). Once the excitation source and/or mechanical compressive force have been removed, the tissue anchor exerts an inward force on the tissue 14 that secures it with slot 16 in a very strong and reliable manner. It has been discovered that by expanding the slot 16 before compression, better fixation forces can be achieved.

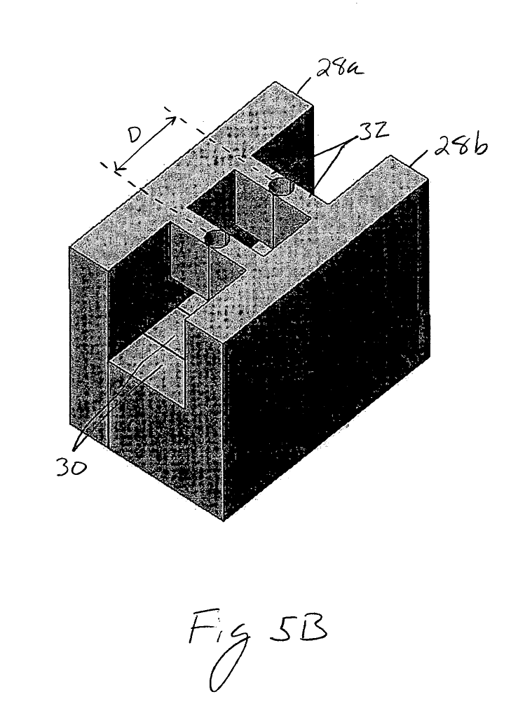

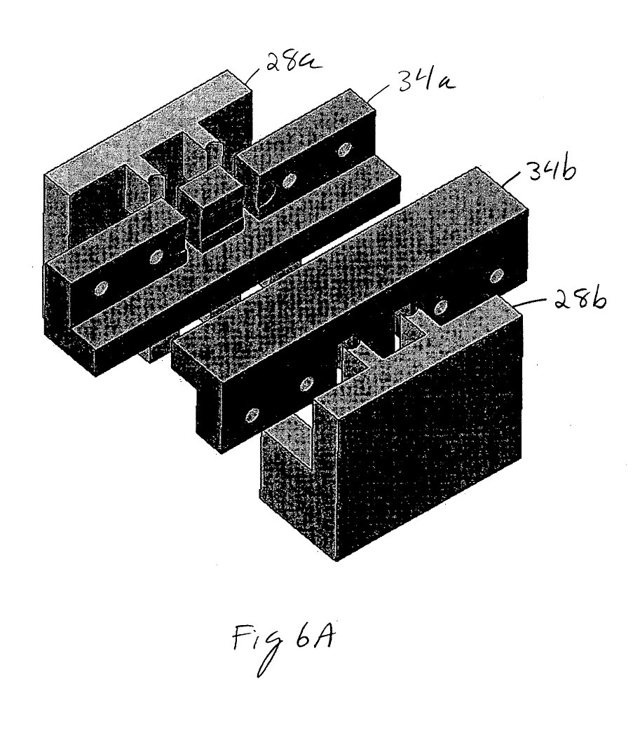

FIGS. 5A and 5B illustrate tooling for compressing the tissue anchors 12. The tooling includes a pair of compression plates 28a/b having engagement tabs 30 and compression tabs 32 extending therefrom. The engagement tabs 30 engage each other to ensure the compression tabs 32 are properly aligned to each other as the plates 28a/b are pressed together. Each of the compression tabs 32 has an end (e.g. preferably concave) to engage with the cylindrical sidewall of the tissue anchors 12. For each compression plate 28a/b, the compression tabs 32 are separated by the distance D. FIG. 5A illustrates the alignment of the plates 28a/b before engagement, and FIG. 5B illustrates the plates 28a/b pressed together.

During the process of compressing the tissue anchors 12 on to the tissue 14, features can be created on the surface of the tissue anchors 12 in the form of ribs, barbs or bumps corresponding to features made in the compression tabs 32. The added features to the tissue anchors 12 would create a positive locking interference fit when pushed into the bone cavity 18.

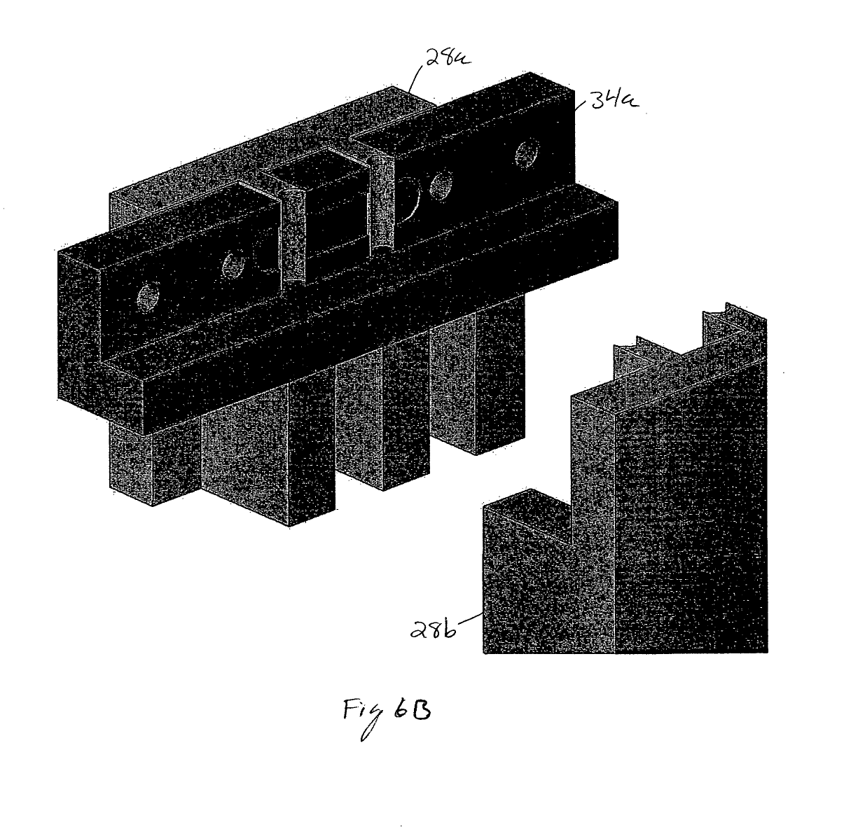

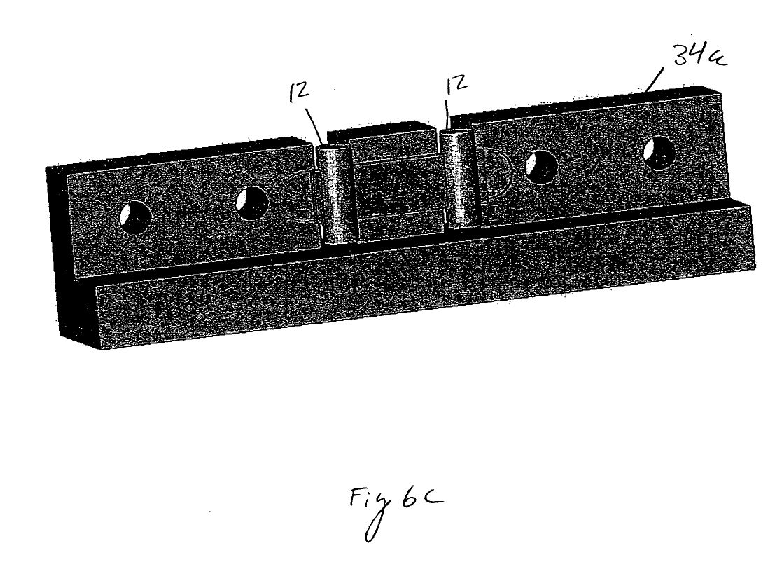

To swage the tissue anchors 12 onto the tissue 14, each tissue anchor is placed between opposing compression tabs 32, and then the plates 28a/b are pressed together so the compression tabs 32 swage (crimp) the tissue anchors 12 down onto the tissue 14. So long as the tissue is pulled taught before or during this process, the tissue anchors 12 are reliably distanced apart along the tissue 14 by the distance D. Interlocking positioning plates 34a/b can be used to reliably position tissue anchors 12 between opposing compression tabs 32, as illustrated in FIGS. 6A to 6D. The compression plates 28a/b can be pressed together using a compression assembly 36 as shown in FIGS. 7A and 7B. The compression assembly includes drive shafts 38 that press the compression plates 28a/b together by the operation of leverage handles 40. Positioning blocks 42 hold the compression plates 28a/b in place. The ends of drive shafts 38 preferably include rotatable bolts 44 that allow the user to adjust the amount of compression exerted on the compression plates 28a/b during the full action of the leverage handles 40.

Bone Cavity Formation

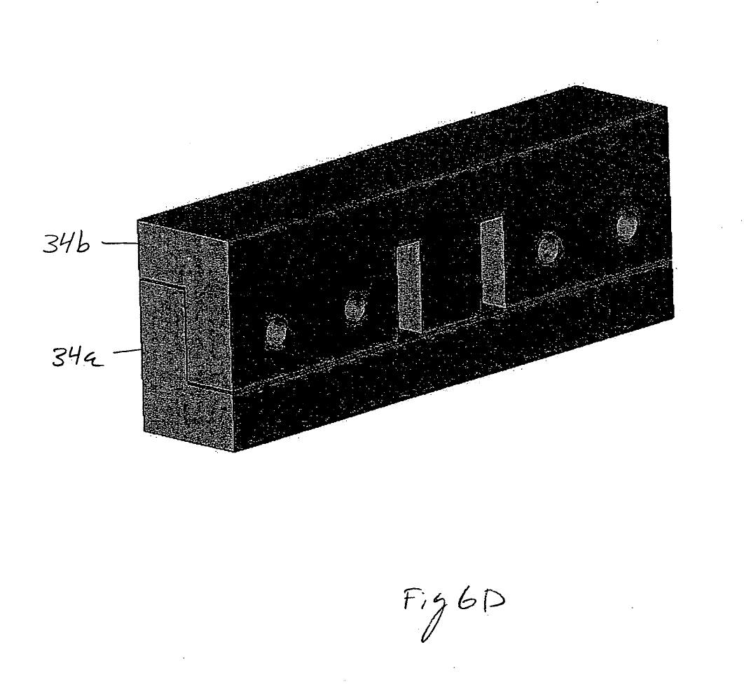

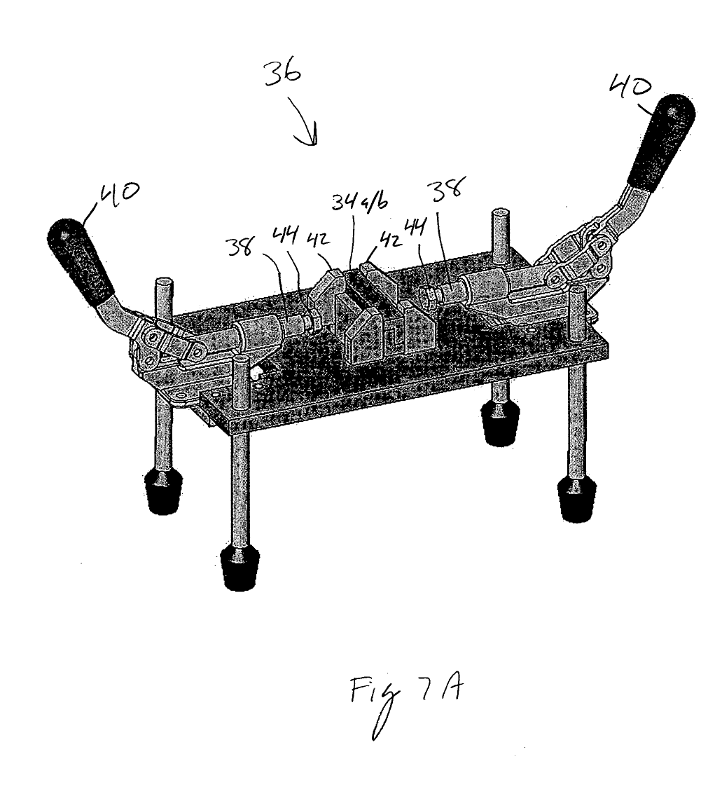

The bone cavity 18 is preferably performed using a drilling template 46 (as shown in FIG. 8A), which is a plate 48 having an aperture 50 formed therethrough with a shape to match the desired dimensions of the bone cavity 18. Forming the bone cavity 18 through the aperture 50 (i.e. using aperture 50 to guide the bone drilling/cutting tools) ensures that the cylindrical holes 22 and channel 24 have the desired dimensions, and holes 22 have the desired separation D. Other aperture(s) and/or slot(s) can be added to the drilling template to help position, or maintain position of, the template 46 on the target bones. Separate apertures for the holes and the channel portions of the bone cavity can be formed on separate inserts, which fit into the template 46 as shown in FIG. 8B.

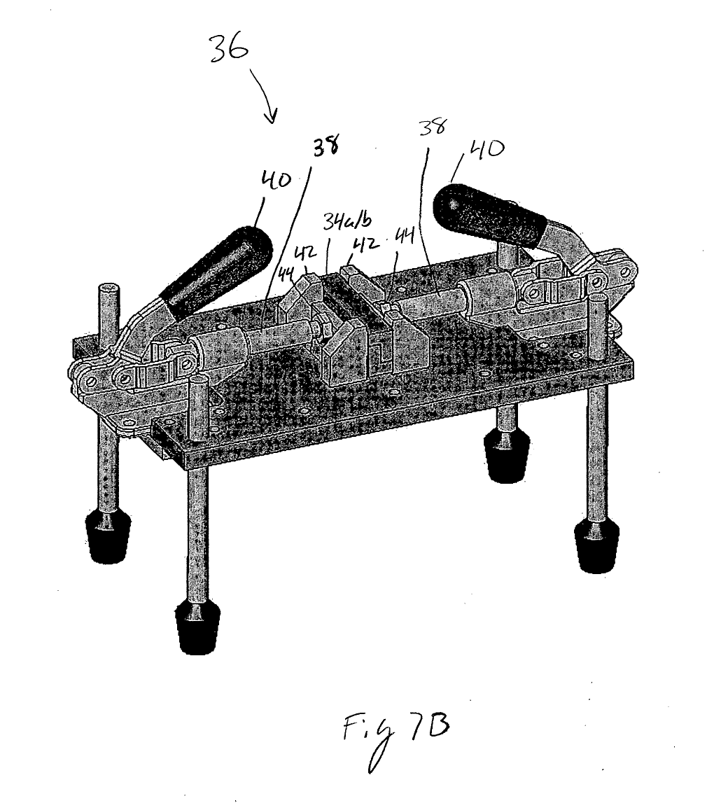

Different matching sets of drilling templates 46 and compression plates 28a/b, with different matching values of separation D, can be provided to the surgeon so that, given the size of the bones to be connected together, the ideal tissue anchor separation value D can be selected and reliably achieved. Thus, the tissue anchor assembly 10 and bone cavity 18 have known and matching shapes. After tissue anchor assembly 10 has been implemented into bone cavity 18 (see FIG. 9), the two bones 20a/b are connected together in a manner that allows motion between the bones during and after the healing process.

The present invention has many advantages: tissue reconstruction is made at or below the interfacing bone surfaces where the original connection tissue existed to encourage re-growth, initial mechanical fixation allows early motion critical for successful healing and preservation of range of motion, and a better means to reliably position and repair the disassociation between bones.

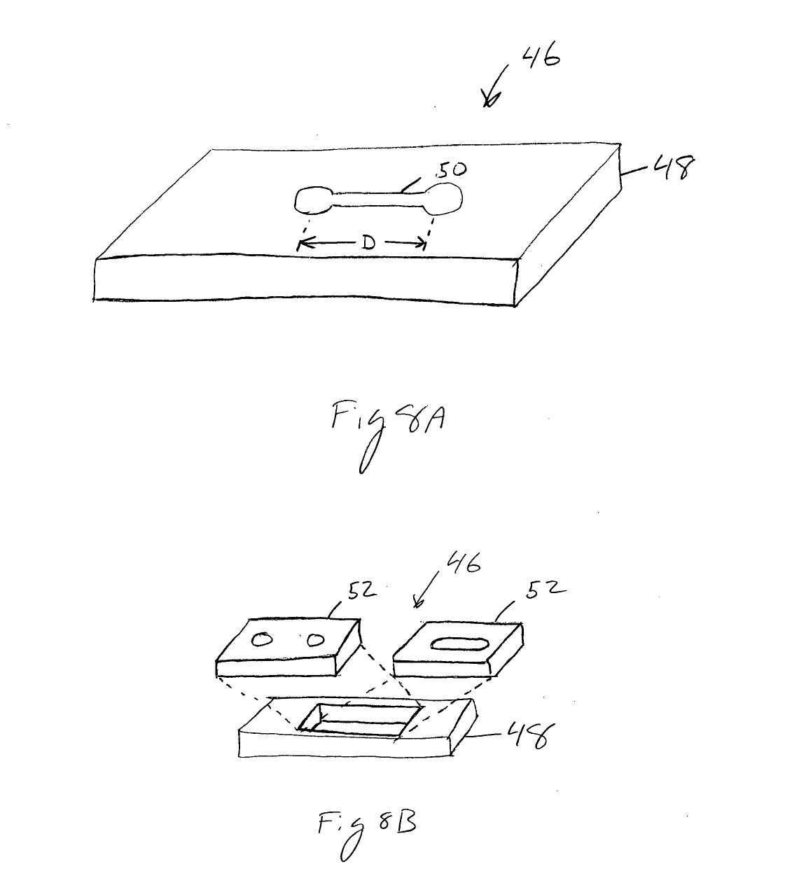

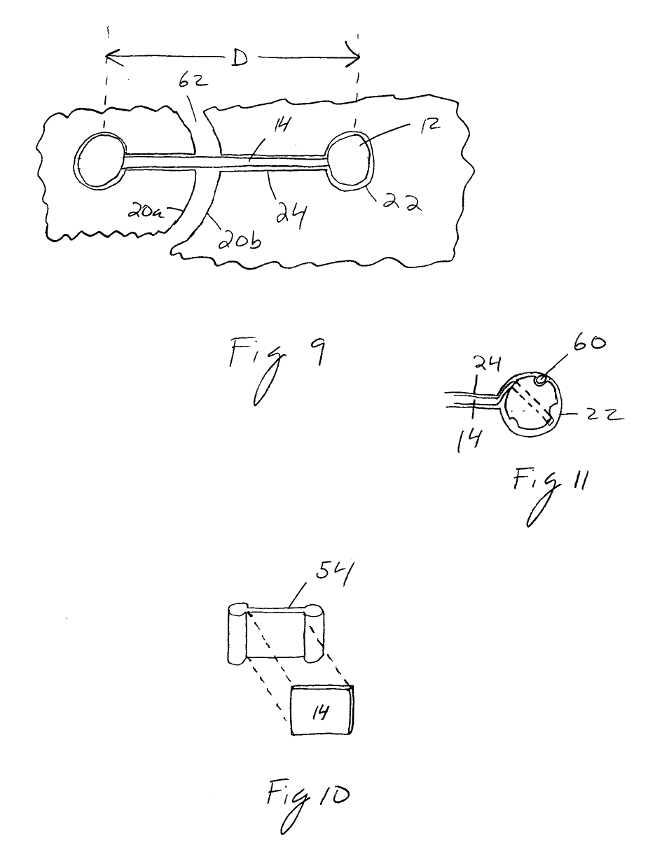

It is to be understood that the present invention is not limited to the embodiment(s) described above and illustrated herein, but encompasses any and all variations falling within the scope of the appended claims. For example, the tissue anchors 12 and bone cavity holes 22 need not necessarily be cylindrical in shape or even the same shape. In order to increase the friction and reduce any possible creep between the tissue anchors 12 and tissue 14, the walls forming slots 16 can be roughened or include a textured pattern (e.g. knurled pattern, tines, pins, intermeshing patterns or grooves, etc.) to better engage with tissue 14. Each tissue anchor could include separate parts or halves that attach together to clamp onto tissue 14, or suture holes so that sutures can be used to help secure the tissue 14 to the tissue anchor 10. Alternately, a tissue carrier 54 could be used, where tissue 14 is attached to one, both sides, or in between layers of the tissue carrier 54 that is shaped to fit the bone cavity, as illustrated in FIG. 10. Lastly, the outer surface of tissue anchors 12 could be irregularly shaped or mismatched in shape in comparison to that of the bone cavity. For example, as shown in FIG. 1, the outer surface is scalloped so that the tissue anchor 12 can be rotated within hole 22 and then fixed in place (e.g. with an interference screw 60 between the tissue anchor surface and the bone) for adjusting (post implementation) the tightness of the joint if necessary. Alternately, other mismatched shapes can be used to ensure there are gaps in which interferences screws can be used to fix rotation (e.g. square anchor in round cavity, etc.

What is claimed is:

1. An anchor system for fixating portions of tissue in a bone cavity, comprising:
a pair of tissue anchors each having an aperture, wherein the aperture includes side walls for receiving and exerting a fixating force onto a portion of the tissue.

2. The anchor system of claim 1, further comprising:
a plate having a drilling aperture formed therein, wherein the drilling aperture has a shape and size that matches that of the pair of tissue anchors and the tissue extending therebetween.

3. The anchor system of claim 2, wherein the tissue anchors each have a cylindrical shape, and wherein each of the apertures have an elongated slot shape extending through the cylindrical tissue anchors.

4. The anchor system of claim 3, wherein the tissue anchors each include a scalloped outer surface.

5. The anchor system of claim 3, wherein the drilling aperture includes a channel portion extending between a pair of cylindrically shaped portions.

6. The anchor system of claim 2, wherein the tissue anchor apertures are formed by excite expansion.

7. An anchor system for securing first and second bones together utilizing tissue having first and second portions and utilizing a bone cavity having a first anchor portion formed in the first bone and a second anchor portion formed in the second bone and a channel extending between the first and second anchor portions, comprising:
a first tissue anchor dimensioned for insertion into the cavity first anchor portion, the first tissue anchor including a first aperture in which the first portion of the tissue is fixated; and a second tissue anchor dimensioned for insertion into the cavity second anchor portion, the second tissue anchor including a second aperture in which the second portion of the tissue is fixated;
wherein the first and second bones are secured together by the first and second tissue anchors inserted in the cavity first and second anchor portions, and by the tissue extending between the first and second tissue anchors and through the channel.

8. The anchor system of claim 7, wherein the first and second tissue anchors each have a cylindrical shape, and wherein each of the first and second apertures have an elongated slot shape extending through the cylindrical tissue anchors.

9. The anchor system of claim 8, wherein the first and second tissue anchors each include a scalloped outer surface.

10. The anchor system of claim 8, wherein the cavity first and second anchor portions each have a cylindrical shape matching the cylindrical shapes of the first and second tissue anchors, respectively.

11. The anchor system of claim 7, wherein the first and second apertures are formed by excite expansion of the first and second tissue anchors.

12. The anchor system of claim 7, wherein the first tissue portion is fixated in the first aperture via excite compression of the first tissue anchor, and wherein the second tissue portion is fixated in the second aperture via excite compression of the second tissue anchor.

13. The anchor system of claim 7, wherein the channel is dimensioned such that sidewalls thereof contact the tissue for forming a biological construct therebetween.

14. The anchor system of claim 7, wherein the first and second tissue anchors each have a shape and dimension that match that of the cavity first and second anchor portions respectively.

15. A method of securing first and second bones together with tissue, comprising:
forming a cavity in the first and second bones, the cavity including a first anchor portion formed in the first bone, a second anchor portion formed in the second bone, and a channel extending between the first and second anchor portions;
fixating a first tissue anchor onto a first portion of the tissue;
fixating a second tissue anchor onto a second portion of the tissue; and
inserting the first tissue anchor into the cavity first anchor portion and the second tissue anchor into the cavity second anchor portion such that the tissue extends along the channel.

16. The method of claim 15, wherein the formation of the cavity includes:
placing a drilling template over the first and second bones, wherein the drilling template includes a drilling aperture formed therein; and
drilling the first and second bones through the drilling aperture to form the cavity.

17. The method of claim 16, wherein:
the drilling aperture includes a pair of cylindrically shaped portions and a channel extending therebetween.

18. The method of claim 15, wherein:
the fixating of the first portion includes forming a first aperture in the first tissue anchor, inserting the first portion in the first aperture, and compressing the first tissue anchor to fixate the first portion in the first aperture; and
the fixating of the second portion includes forming a second aperture in the second tissue anchor, inserting the second portion in the second aperture, and compressing the second tissue anchor to fixate the second portion in the second aperture.

19. The method of claim 18, wherein:
the formation of the first aperture includes excite expanding the first tissue anchor; and
the formation of the second aperture includes excite expanding the second tissue anchor.

20. The method of claim 18, wherein:
the compressing of the first tissue anchor includes excite compressing the first tissue anchor; and
the compressing of the second tissue anchor includes excite compressing the second tissue anchor.

21. The method of claim 15, wherein the channel is dimensioned such that sidewalls thereof contact the tissue for forming a biological construct therebetween.
